(19) United States
(12) Patent Application Publication
(10) Pub. No.: US 2009/0069734 A1
(43) Pub. Date: Mar. 12, 2009

(54) Flextender

(76) Inventor: Reesa Holland, Midland, TX (US)

Correspondence Address: JAMES RAY & ASSOCIATES, 2640 PITCAIRN ROAD, Monroeville, PA, 15146, US

(21) Appl. No.: 12/228,987

(22) Filed: Aug. 19, 2008

Related U.S. Application Data
(60) Provisional application No. 60/970,728, filed on Sep. 7, 2007.

(51) Int. Cl. A61F 5/00

(52) U.S. Cl. 602/22

(57) ABSTRACT

An apparatus for separating fingers and allowing a user the ability to exercise fingers when recovering from an arm or a hand injury. Such apparatus is designed to keep the fingers straight as possible yet permitting the fingertips to flex. The apparatus made of a foam type material has projections that fit between the fingers and have cut out portions that fit snugly on the knuckles that are farthest from the nails. The cutouts engage the four fingers with the thumb remaining loose. Thus, the fingers are supported in an outstretched manner.

### CROSS REFERENCE TO RELATED APPLICATIONS

This application is closely related to and claims benefit from U.S. Provisional Application Ser. No. 60/970,728 filed Sep. 7, 2007

### FIELD OF THE INVENTION

The present invention relates, in general, to an apparatus for persons healing from an arm or hand injury, and, more particularly, the present invention relates to an apparatus that keeps fingers extended and separated during the healing process and also permitting the fingers to wiggle or exercise the fingers in order to prevent stiffening.

### BACKGROUND OF THE INVENTION

The natural tendency during movement is to carry the arm close to the body and curl or cup the fingers which is a comfortable position. However, it is not always a good position for a patient who must concentrate on finger movement. For one who has suffered an arm or hand injury, trying to extend the curled fingers can be extremely painful. Lengthy periods of curling the hand and fingers reduce circulation and causes stiffness to set in. Any progress in stretching and/or exercising the fingers is set back once the fingers are allowed to relax back into that “comfortable” curled position. Without keeping the fingers flexible though, the muscles can become weak causing the need for strength training after the main injury has healed.

Thus, it would be advantageous if there were a simple and effective means for such a patient to keep the fingers spread and flexible and not in a curled position.

### SUMMARY OF THE INVENTION

In a first aspect the present invention provides an apparatus for separating fingers and allowing a person the ability to exercise fingers when recovering from one of an arm and a hand injury. Such apparatus comprises a supporting member having a predetermined shape and formed of a predetermined material. A plurality of projecting members are formed on the supporting member, such plurality of projecting members includes three inner projecting members having a concave portion formed on each side of the three inner projecting members closely adjacent such supporting member and having a bulbous portion on an end of the three projecting members that is farthest from the supporting member. The plurality of projecting members includes a first member of the three inner projecting members for engagement with an inner portion of a first finger and a corresponding inner portion on a first side of a middle finger. A second member of the three inner projecting member for engagement with an inner portion of a first side of a ring finger and a corresponding inner portion of a second side of such middle finger. A third member of the three inner projecting members for engagement with an inner portion of a second side of such ring finger and an inner portion of a little finger. There are two outer projecting members, each of the two outer projecting members having a concave portion on an inner surface thereof closely adjacent the supporting member and a half of a bulbous portion disposed on an inner portion of an end of the two outer projecting members farthest away from such supporting member. A first member of the two outer projecting members is for engagement with an outer portion of such first finger and a second member of such outer projecting members is for engagement with an outer portion of such little finger.

In a second aspect the present invention provides a method for each of exercising fingers and for maintaining fingers separated during rehabilitation from one of a hand and an arm injury. The method comprises the steps of extending fingers of a hand during healing from an injury. There is a step of placing a specially designed apparatus having a plurality of projections between the fingers and having a cut out portion on the plurality of projections for fitting over knuckles of the hand for maintaining the fingers of the hand separated and a step of exercising the fingers for a predetermined period of time in order to prevent stiffening of such finger joints.

### OBJECTS OF THE INVENTION

It is, therefore, one of the primary objects of the present invention to provide an apparatus for keeping fingers as straight as possible.

Another object of the present invention is to provide an apparatus for keeping fingers straight by permitting the finger tips to be wiggled or flexed.

Still another object of the present invention is to provide an apparatus for keeping the fingers separated and extended while healing from a hand or arm injury.

In addition to the various objects and advantages of the invention which have been described in some specific detail above it should be noted that various other objects and advantages of the present invention will become more readily apparent to those persons who are skilled in the relevant art from the following more detailed description, particularly when such description is taken in conjunction with the appended claims.

### BRIEF DESCRIPTION OF THE DRAWINGS

FIG. 1 is a top perspective view of the apparatus according to an embodiment of the invention.

FIG. 2 is a front perspective view of the apparatus shown in FIG. 1.

FIG. 3 is a top perspective view of the apparatus shown in FIG. 1 as used to separate the fingers of a hand.

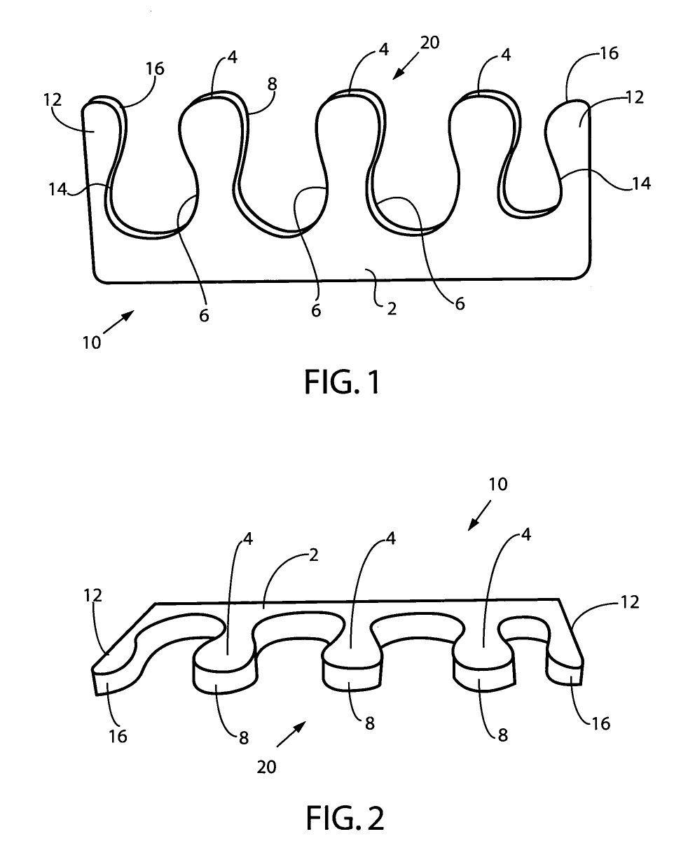

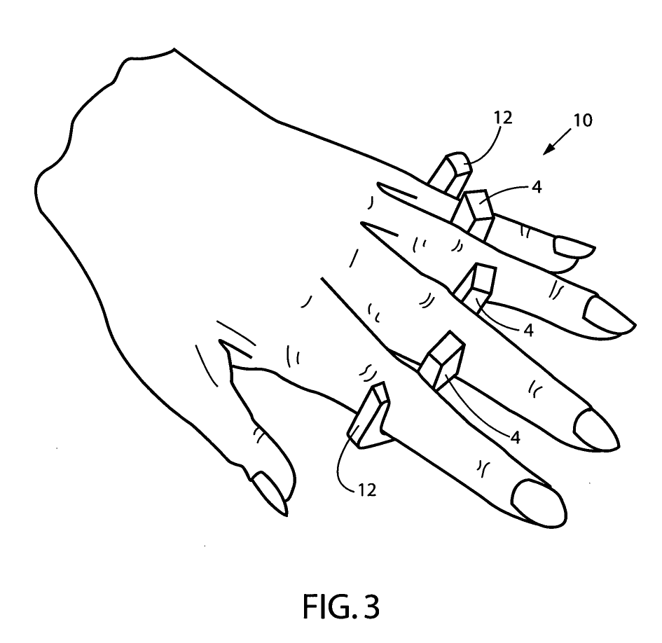

### BRIEF DESCRIPTION OF THE PRESENTLY PREFERRED AND VARIOUS ALTERNATIVE EMBODIMENTS OF THE INVENTION

Prior to proceeding with the more detailed description of the present invention it should be noted that, for the sake of clarity, identical components which have identical functions have been designated by identical reference numerals throughout the several views illustrated in the drawings.

In a first aspect the present invention provides an apparatus, generally designated 10, for separating fingers and allowing a person the ability to exercise fingers when recovering from one of an arm and a hand injury. Such apparatus 10 comprises a supporting member 2 having a predetermined shape and formed of a predetermined material. A plurality of projecting members 20 are formed on the supporting member 2, such plurality of projecting members 20 includes three inner projecting members 4 having a concave portion 6 formed on each side of the three inner projecting members 4 closely adjacent such supporting member 2 and having a bulbous portion 8 on an end of the three projecting members 4 that is farthest from the supporting member 2. The plurality of projecting members 20 includes a first member of the three inner projecting members 4 for engagement with an inner portion of a first finger and a corresponding inner portion on a first side of a middle finger. A second member of the three inner projecting members 4 for engagement with an inner portion of a first side of a ring finger and a corresponding inner portion of a second side of such middle finger. A third member of the three inner projecting members 4 for engagement with an inner portion of a second side of such ring finger and an inner portion of a little finger. There are two outer projecting members 12, each of the two outer projecting members 12 have a concave portion 14 on an inner surface thereof closely adjacent the supporting member 2 and a half of a bulbous portion 16 disposed on an inner portion of an end of the two outer projecting members 12 farthest away from such supporting member 2. A first member of the two outer projecting members 12 is for engagement with an outer portion of such first finger and a second member of such outer projecting members 12 is for engagement with an outer portion of such little finger.

Such predetermined material for forming such support member 2 is selected from foam rubber and foam plastic materials. It is also preferred that such supporting member 2 and such plurality of projecting members 20 are formed of a similar material and further that such supporting member and such plurality of projecting members 20 are formed as a single unit.

Such concave portions 6,14 on the plurality of projecting members 20 are formed to fit snugly on knuckles of such fingers farthest from a finger nail.

Such supporting member 2 is sufficiently flexible that such support member 2 can bend sufficiently that such apparatus 10 can engage such knuckles on all four fingers.

Such supporting member 2 and such plurality of projecting members 20 keep such fingers straight and separated in an extended position while permitting wiggling and exercising such fingers.

A diameter of a circular indentation formed by two adjacent concave portions 6,14 for each of the plurality of projecting members 20 for engagement with an index, middle and ring finger is between about ⅝ and about ⅞ inches.

The diameter of the circular indentation formed by two adjacent concave portions 6,14 for each of such plurality of projecting members 20 for engagement with such index, middle and ring finger is about ¾ inch.

The diameter of a circular indentation formed by two adjacent concave portions 6,14 for engagement with such small finger is between about ⅜ and about ⅝ inches and preferably that such diameter is substantially ½ inch.

In a second aspect the present invention provides a method for each of exercising fingers and for maintaining fingers separated during rehabilitation from one of a hand and an arm injury. The method comprises the steps of extending fingers of a hand during healing from an injury. There is a step of placing a specially designed apparatus having a plurality of projections between the fingers and having a cut out portion on the plurality of projections for fitting over knuckles of the hand for maintaining the fingers of the hand separated and a step of exercising the fingers for a predetermined period of time in order to prevent stiffening of such finger joints.

The method includes a step of keeping the fingers straight and preventing curling of the fingers. It should be noted that such apparatus is formed of a foam material which is sturdy but flexible enough that it can fit over the large knuckles of the four fingers thereby keeping the fingers straight. There is further the step of exercising the fingers includes wiggling the tips of the fingers.

While a presently preferred embodiment and alternate embodiments of the present invention have been described in detail above, it should be understood that various other adaptations and/or modifications of the invention can be made by those persons who are particularly skilled in the art without departing from either the spirit of the invention or the scope of the appended claims.

I claim:

1. An apparatus for separating fingers and allowing an ability to exercise fingers when recovering from one of an arm and a hand injury, said apparatus comprising:
(a) a supporting member having a predetermined shape and formed of a predetermined material;
(b) a plurality of projecting members formed on said supporting member of said predetermined material, said plurality of projecting members including three inner projecting members having a concave portion formed on each side of said three inner projecting members closely adjacent supporting member and having a bulbous portion on an end of said three projecting members that is farthest from said supporting member, said plurality of projecting members including;
(i) a first member of said three inner projecting members for engagement with an inner portion of a first finger and a corresponding inner portion on a first side of a middle finger;
(ii) a second member of said three inner projecting member for engagement with an inner portion of a first side of a ring finger and a corresponding inner portion of a second side of such middle finger;
(iii) a third member of said three inner projecting members for engagement with an inner portion of a second side of such ring finger and an inner portion of a little finger; and
(iv) two outer projecting members, each of said two outer projecting members having a concave portion on an inner surface thereof closely adjacent said supporting member and a half of a bulbous portion disposed on an inner portion of an end of said two outer projecting members farthest away from said supporting member, a first member of said two outer projecting members for engagement with an outer portion of such first finger and a second member of said outer projecting members for engagement with an outer portion of such little finger.

2. The apparatus, according to claim 1, wherein said predetermined material for forming said support member is selected from foam rubber and foam plastic materials.

3. The apparatus, according to claim 1, wherein said concave portions of said plurality of projecting members are formed to fit snugly on knuckles of such fingers farthest from a finger nail.

4. The apparatus, according to claim 3, wherein said supporting member is sufficiently flexible that said support member can bend sufficiently that such apparatus can engage such knuckles on all four fingers.

5. The apparatus, according to claim 1, wherein said supporting member and said plurality of projecting members keeps such fingers straight and separated in an extended position and permits wiggling and exercising such fingers.

6. The apparatus, according to claim 1, wherein a diameter of a circular indentation formed by two adjacent concave portions for each of said plurality of projecting members for engagement with an index, middle and ring finger is between about ⅝ and about 7/8 inches.

7. The apparatus, according to claim 6, wherein said diameter of said circular indentation formed by two adjacent concave portions for each of said plurality of projecting members for engagement with such index, middle and ring finger is about ¾ inch.

8. The apparatus, according to claim 1, wherein a diameter of a circular indentation formed by two adjacent concave portions for engagement with such small finger is between about ⅜ and about ⅝ inches.

9. The apparatus, according to claim 8, wherein said diameter of said circular indentation formed by said two adjacent concave portions for engagement with such small finger is about ½ inch.

10. The apparatus, according to claim 1, wherein said supporting member and said plurality of projecting members are formed of a similar material.

11. The apparatus, according to claim 1, wherein said supporting member and said plurality of projecting members are formed as a single unit.

12. A method for each of exercising fingers and for maintaining fingers separated during rehabilitation from one of a hand and an arm injury, said method comprising the steps of:
(a) extending fingers of a hand during healing from an injury;
(b) placing a specially designed apparatus having a plurality of projections between said fingers and having a cut out portion on said plurality of projections for fitting over knuckles of said hand while maintaining said fingers of said hand separated; and
(c) exercising said fingers for a predetermined period of time in order to prevent stiffening of such finger joints.

13. The method, according to claim 12, wherein said method includes a step of keeping said fingers straight and preventing curling of said fingers.

14. The method, according to claim 12, wherein said exercising includes a step of wiggling fingertips of said fingers.
